(19) United States
(12) Patent Application Publication
(10) Pub. No.: US 2024/0400956 A1
(43) Pub. Date: Dec. 5, 2024

(54) PRODUCTION EQUIPMENT AND FACILITIES FOR UNICELLULAR MICROORGANISMS, MICROORGANISM COMMUNITIES, MULTICELLULAR PLANT AND ANIMAL CELLS AND AQUATIC ORGANISMS

(71) Applicant: Mood Harvest AS, Oslo (NO)

(72) Inventor: Fredrik MOOD, Oslo (NO)

(21) Appl. No.: 18/576,138

(22) PCT Filed: Jul. 4, 2022
(86) PCT No.: PCT/NO2022/050161

§ 371 Date: Jan. 3, 2024

(30) Foreign Application Priority Data
Jul. 5, 2021 (NO) 20210868

(51) Int. Cl. C12M 1/00; C12M 1/09; C12M 1/12

(52) U.S. Cl. CPC C12M 21/02; C12M 23/06; C12M 23/56; C12M 31/00; C12M 43/06; C12M 47/10

(57) ABSTRACT

Production equipment for production of biological material, comprising a bioreactor 12, 3 for the cultivation and production of unicellular microorganisms and communities of microorganisms as well as multicellular aquatic plant and animal organisms. The bioreactor has at least one space for the proliferation 21 of unicellular microorganisms, communities of microorganisms, multicellular aquatic plants and animals, which space is in communication with a device 23 for cultivating, transporting and harvesting said microorganisms or aquatic organisms, comprising a pipe chain conveyor.

### FIELD OF THE INVENTION

The present invention relates to at least one bioreactor and a facility for production of unicellular microorganisms, communities of microorganisms, plant and animal cells in culture and aquatic organisms as well as the use of the installation for the production of proteinaceous material and possibly for the production of biologically active elements such as hormones, enzymes and structural proteins.

### BACKGROUND

The world is experiencing significant population growth and soon the planet will reach 10 billion people. The global need for protein will rise in line with the growing population and according to the UN; the world food production will increase 70% by 2050. A major undertaking going forward is to ensure that enough food are produced for a growing population and for that, we must make sure new methods and systems are developed allowing us to produce large quantities of sustainable protein in an industrial and climate-neutral way.

The EU countries, Norway and countries in Asia currently have protein deficit. This means that these countries must import protein-rich feed as raw materials for their own fish- and concentrated-feed production. In this context, large quantities of Soya are imported.

With increasing demand for soya protein that are cheap to produce, the production of soya has experienced tremendous growth. The UN have estimated that soya consumption will double in the next fifty years. A growth that most likely have to happen at the expense of rainforests and other vulnerable ecosystems.

Prohibiting the soya industry to grow is not without problems, as this may lead to increased prices on soya products, which in turn will make it more expensive to produce protein such as meat and fish. A way around is to put in place a full-fledged alternative for soya products. Single-cell protein or similar protein production can be such an alternative and can play an important role. The fact that the Nobel Peace Prize awarded in 2020 the World Food Program (WFP) underlines the importance access to good and nutritious food is, and will be for the future.

With constant innovations in biotechnology and biology, there is also a need to produce protein-based biologically active compounds that can be used in the pharmaceutical industry. Production factories that are based on microorganisms of both prokaryotic and eukaryotic character are in demand to make sufficient quantities of such proteins such as hormones, enzymes and/or structural proteins such as human insulin, human growth hormone, human adrenaline etc . . . In such production, both naturally occurring and genetically engineered microorganisms are used.

It is previously known to use single-cell organisms to produce single-cell protein. Single-cell protein is protein that is extracted from single-celled organisms, such as bacteria, algae and fungal species, which are grown in various nutrient substrates, for example fractions of petroleum or waste products from the cellulose industry.

Single-cell protein is well known, and several protein products are commercially available and approved. Several products for raw materials for fish feed are in fact approved.

BioProtein as an example is EU approved to be use in fish feed for salmon, with admixture of up to 19% for fish in fresh water and 33% in salt water. BioProtein is also EU-approved with up to 8% in feed for pigs from 25 to 60 kg, and with 8% in feed for calves over 80 kg.

Bioprotein is produced by continuous fermentation with natural gas as an energy and carbon source and ammonia as a nitrogen source, as well as mineral salts that are added in the process. The biomass mainly consists (95%) of the aerobic methanotrophic bacterium Methylococcus capsulatus (Bath). BioProtein has a dry matter content of approx. 95% and contains approx. 70% crude protein and 10% fat. The protein has a favorable amino acid composition with a high content of tryptophan, but a somewhat lower content of lysine than what is found in fishmeal.

Trials have shown that protein and amino acids from BioProtein are digested well by pigs, chickens, mink and salmon. The use of BioProtein as a substitute for protein from fishmeal in feed for fish, or as a substitute for fishmeal and soymeal in feed for chickens and pigs, has shown good results with regard to growth, feed intake and feed utilization. Source: http://www.umb.no/stattik/husdyrforsoksmoter/2009/5.pdf'

Research is currently being done into the possibility of using modern genetic technology and metabolic manipulation to change and or insert new genes so that bacteria convert as much as possible of the relevant raw material (for example methanol) into the desired product (potein/lysine). Among other things, research on the bacterium Bacillus methanolicus, which known to produce small amounts of lysine, is done. There is also a lot of research into E. coli bacteria and how to modify these to consume more CO₂ than they release.

Omega-3 fatty acids in the fish farming industry comes from fish oil, but with more and more global shortage of this oil, alternative sources must be developed. One possibility is to cultivate microalgae with a high omega-3 content in a controlled environment such as in a photo-bioreactor. These microalgae grow in water substrate and use CO₂ as a carbon source in photosynthesis and energy from light. Microalgae can contain many other important nutrients, such as antioxidants, vitamins and minerals. Examples of microorganisms that can produce omega-3 fatty acids such as EPA (eicosapentaenoic acid) and DHA (docosaheptaenoic acid) are Schewanella putrefaciens, Alteromonas putrefaciens, Pneumatophorus japonicus, Photobacterium, Thraustochytrium aurenum, Mortierella, Phytium, and Phytium irregulare. Important vitamins such as vitamin B12 and vitamin C can also be produced by microorganisms.

In 2018, Norway produced 1.2 million ton of salmon, to a value of NOK 64.6 Billion. In the same period, 1.8 million ton of fish feed were produced. Approximately 300-400,000 ton of soya are imported each year into Norway. As most of the raw material for the fish feed produced today has to be imported, there is clearly a need to ensure own local production of protein, mono- and polyunsaturated fatty acids, carbohydrates and amino acids.

A production facility that the application relates to can have the capacity to produce a significant proportion of what is currently imported.

### Example: Single-Cell Protein Fermenter and Photo Bioreactor Equilibrium

| Quantity per year | Bioreactor | Photobioreactor |
|---|---|---|
| Volume [m³] | 1200 | 80000 |
| Methane [mT] | 63072 | — |
| Oxygen [mT] | 210240 [symbol] | [symbol] 126144 |
| Carbon dioxide [mT] | 210240 [symbol] | [symbol] 210240 |
| Air [mT] | 630720 | — |
| Ammonia [mT] | 10512 | — |
| SCP [mt]/Algea [mt] | 47304 | 84096 |

The content of the salmon feed that today is imported we can probably replace, with local produced protein and omega 3 from bacteria and algae. Such protein and omega3 products have great potential, but must become more cost competitive if to replace soya. The today's production methods are not all climate neutral and there are not many facilities if any that can produce the volumes in question profitably.

There is clearly a need for a sustainable and climate-neutral ways to produce large amounts of protein and omega3 industrially. There is also a need for developing fermenter and bioreactor systems for industrial scale production.

Some researchers claim that within a few decades we will have run out of phosphorus. This may have catastrophic consequences for the world's food production. One can already see today that the marked for phosphorus has intensified and that the price of the mineral, used in fertilizer throughout the world, has doubled sevenfold in just a few years. Developing effective methods for producing protein from phosphorus sources will be important in the future. As an example, one can recover phosphorus from the farming of fish and land animals and use it for fish or animal feed. Another source where large amounts of phosphorus are emitted is from sewage and water treatment plants. It is very likely that in the future one will get greater demand for reducing emissions of phosphorus. The technical solution described herein will be able to harvest and make use of this phosphorus in a circular way.

The Paris Agreement, ratified by Norway and plural of other countries in 2015; states that the countries of the world must strive to keep global warming well below two degrees and preferably down to one and a half degrees in order to limit climate change.

Here are some of the consequences, if we cannot reverse this trend:
99% of coral reefs disappear (70-90 percent with 1.5° C.)
The permafrost defrosts more (28-53 percent with 2° C. compared to 17-44 percent with 1.5° C.)
More extreme heat—more intense heat waves.
More forest fires, such as those that occurred in California in the fall of 2018
Heavier precipitation where precipitation increases, especially in connection with tropical storms
Longer and more intense drought periods, such as occurred in Norway in the summer of 2018
Increased risk of the ice belt in Greenland and Antarctica collapses—and a very high probability of an ice-free Arctic in the summer
Higher risk of heat-related illnesses and deaths
Ocean acidification is increasing considerably, and thus the vulnerability of many species in the sea

### Source: 1.5° C.: What does it mean?—Energy and Climate

In order to achieve the goal of becoming climate neutral in 2050-2100, we cannot allow ourselves to develop production processes where there are large emissions of greenhouse gases such as methane (CH₄) and carbon dioxide (CO₂) to name a few. Methane CH₄ is a very powerful greenhouse gas, and it contributes to solar energy being stored in the atmosphere and causes the temperature to rise. CH₄, which contributes to the greenhouse effect, is 22 times more effective than carbon dioxide (CO₂).

One tool to reduce CO₂ emissions is to introduce tax on emissions. Today there is a CO₂ tax of NOK 590/ton for industry subject to quotas. This tax is expected to increase significantly in the coming years in order to financially stimulate emission cuts. Industries not subjected to quotas will in future most likely have to commit to CO₂ cuts and can expect CO₂ taxes.

Consequently, there is a need for a climate-neutral way to produce protein- and oil-rich organisms that can form basis for the further production of feed and special products.

The difference between a bioreactor and a fermenter is the type of biochemical reaction that takes place inside the closed vessel those such systems comprises. A bioreactor enables all types of biochemical reactions, but a fermenter only deals with fermentation. In simple terms, it said that fermentation operates under anaerobic (without oxygen) conditions, while bioreactors can operate/run under both aerobic (with oxygen) and anaerobic (without oxygen) conditions.

The various processes enable rapid generation success, for algae 2-6 hours, for yeast 1-3 hours, for bacteria 0.5-2 hours.

With increased population growth and climate challenges, there may be a shortage of land available for industrial purposes. As is well known, the Earth's surface consists of 30 percent land and 70 percent sea. Better utilization of sea areas will therefore be a necessity in the future.

Consequently, there is a need to develop production methods and units that one can used on large bodies of water such as the sea.

Known technologies that may be useful in understanding the background include:

Norwegian patent application WO03016460A1, which describes a method for the production of biomass by cultivating a microorganism in an aqueous liquid culture medium that circulates in a loop reactor with a degassing zone for the emission of gas where carbon dioxide-containing waste gas is removed from the reactor and upstream a degassing zone where a propellant gas is introduced to drive carbon dioxide in the liquid phase into a repairable discharge gas phase and has upstream of the degassing zone a nutrient gas introduction zone, where oxygen is introduced into the reactor and mixed with the liquid culture medium therein, characterized in that oxygen introduction into the nutrient gas introduction zone is carried out at several places along the flow path through the loop reactor at a rate such that the average dissolved oxygen content of the liquid culture medium measured using a polygraphic oxygen electrode does not exceed 25 ppm.

Danish patent application WO0070014 A8

A fermenter and a fermentation method in a U-shaped fermenter comprising a U-portion having a substantially vertical downflow portion, a substantially vertical upflow portion, and a substantially horizontal connecting portion, connecting the lower ends of the downflow portion and the upflow portion, a top portion provided above the U-section and has a diameter that is significantly larger than the diameter of the U-section, and which is designed to create liquid circulation in the U-section of the fermenter, and one or more gas injection points for the introduction and dispersion of the gases in the fermentation liquid. The pressure can be controlled differently in specific zones in the fermenter by pressure regulating devices, e.g. by increasing the pressure in certain zones of the fermenter relative to the pressure in other zones of the fermenter, or reducing the pressure in one zone of the fermenter relative to the pressure in another zone of the fermenter.

US Patent Application U.S. Pat. No. 4,116,778A

Describes a facility for the continuous cultivation of microorganisms. The plant consists of a closed recirculation circuit consisting of a fermentation device, a pump and a process parameter measuring unit connected in series by means of a channel. The plant also consists of a container for storing a nutrient medium and a finished product collector with a supply line and an overflow connection. Downstream of the measuring unit, the annular channel has two locking devices; connected to the part of the channel between the locking devices is the supply line and the overflow connection, which alternately account for equal volumes of a nutrient medium introduced into the recirculation circuit and of a suspension of microorganisms which are simultaneously discharged therefrom.

Norwegian patent application NO20100465

Describes tanks and bulk carriers with tanks that can be converted into breeding facilities for aquatic organisms. Such a closed facility achieves a well-controlled farming environment/facility physically separated from the sea, where disadvantages related to escape, salmon lice, disease and emissions do not pose a problem.

International publication WO2016060892A1

Describes an algae cultivation system with a passive membrane photobioreactor that has an inner space in which algae can be grown and a porous membrane that separates growth media from the inner space, where water, carbon dioxide and nutrients contained in the growth medium can pass through the membrane and into the inner space, but pollutants cannot. A belt system consisting of a porous membrane which separates an inner and an outer space and which can be scraped with blades and lead nutrient medium inside an open unit is also described.

It is known to use MBR systems for cleaning and breaking down biological waste on board ships (cruise ships, ferries, FPSOs, etc.) before any discharge to sea.

### GENERAL DESCRIPTION OF THE INVENTION

In one embodiment of the invention, at least one bioreactor is provided. The bioreactor can, but is not limited to, be placed on a floating production unit. The production unit will then have equipment for the production of unicellular microorganisms, microorganism communities, multicellular plant and animal cells and aquatic organisms, where the production unit includes at least one floating unit. It can be a new build, or a converted tank-, bulk-, chemical-ship, barge, catamaran, raft, support vessel, semi-submersible semi/rig, breeding cages and breeding structures or similar. The production unit further comprises at least one fermentation reactor and/or at least one bioreactor, both with associated pumps and/or chain conveyors, valves, branch pipes and pipelines that regulate quantity and fluid level and filling and emptying.

In order to make the best use of the hull deck area and tank volume, when the floating unit is a ship. One embodiment can be to place fermentation units on deck with pipe loops going down into the hull and placing bioreactors within the hull with pipe loop stretching up above deck.

Another embodiment can be self-floating and/or submersible reactors that connects to a support unit via flexible hoses and cables. These units can be equipped with solar panels.

Another embodiment describe a photo-bioreactor with internal moving lights and integrated filtration and continuous or intermittent harvesting system. The photo bioreactor can be open systems and or closed systems. The reactor is not limited to a floating production unit and can be placed on land or in the sea.

One or more external breeding units with a ballast system are described in another embodiment, which are attached to the production vessel with one or more running cats and winch systems.

The production unit may also include a system for separating and processing the products from the various processes. Examples of such systems are skimmers that separate and/or remove foam and liquid material on the surface of the bioreactors. Filters or filter systems through which the liquid in the bioreactors are filtered, both to clean the liquid in the bioreactors and to collect material from unicellular organisms that can form a starting material for the food and feed additive according to the invention. It may also be relevant to decant liquid from the bioreactor and/or fermenter to separate solids from liquid.

The production plant according to the invention is in one embodiment preferably designed so that the purification units for the bioreactor are adapted to purify at least 10% of the exhaust gases from the fermentation reactor.

The features described in claims 1 to 16 characterize production equipment and facilities. The attached dependent requirements specify alternative and/or advantageous designs.

### BRIEF DESCRIPTION OF THE FIGURES

Examples of embodiments according to the present inventions will now be described with reference to the attached figures, where:

FIG. 1 shows a schematic diagram of a production unit seen from the side,

FIG. 2 shows a principle sketch of the production unit shown in FIG. 1 top view,

FIG. 3 shows principle sketches of the production unit shown in FIG. 1 transom,

FIG. 4 shows a flow chart of a production process type 1,

FIG. 5 shows a schematic diagram of a photobioreactor with pipe chain conveyor

FIG. 6 shows a schematic diagram of a fermentation reactor with pipe chain conveyor

FIG. 7 shows a schematic diagram of a reactor with branch pipes

FIG. 8 shows a principle sketch of a self-floating and or submersible bioreactor which is connected to a support vessel.

FIG. 9 shows a schematic diagram of a production unit with rearing cages set from above.

FIG. 10 Shows a principle sketch of the production unit shown in FIG. 9 cross-ship, and shows normal, intermediate and protected condition

FIG. 11 Shows a principle sketch of the production unit shown in FIG. 9 cross-ship, and how the farming unit is operated.

FIG. 12 shows a schematic diagram of the attachment of the rearing unit to the hull of the floating device.

### DETAILED DESCRIPTION

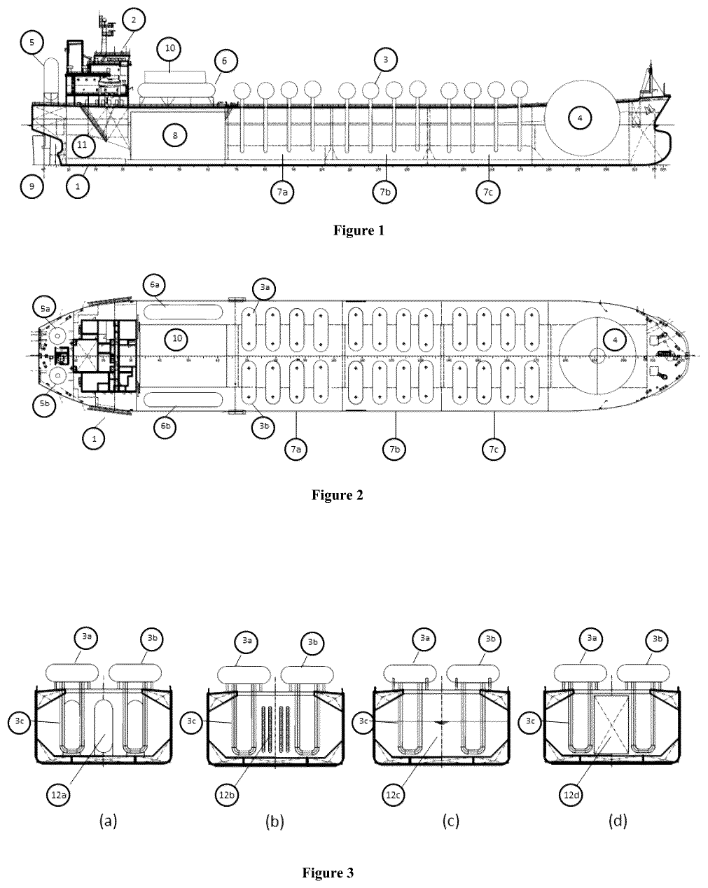

FIG. 1 and FIG. 2 show principle sketches of an embodiment seen from the side and from above, showing a floating production unit 1 for unicellular microorganisms and communities of microorganisms, multicellular plants and animals and aquatic organisms. Here is shown, for example, a setup comprising a cargo ship with 24 fermentation units 3, distributed over 8 units per hold 7a-b, a process module 10 linked to drying and processing as well as storage tanks 4,5,6,7,8 for storing various nutrient substrates but not limited to (O₂, CO₂, NH₃, NH₄, H₂, P), minerals and bulk. Shown are also two ammonia tanks 5a, b aft of the wheelhouse 2. Two oxygen tanks 6a, b forward of the wheelhouse. A tank 4 in the hold closest to the bow, where natural gas (methane, propane, ethane, butane) can either be stored in liquid or gaseous form and can be of independent or integrated type and a large bulk tank 8. In an alternative embodiment, natural gas can come from an external source. The volume occupied by the tank are replaced with additional fermentation 3 and bioreactor 12 units. The superstructure 2 has necessary facilities such as cabins, offices, mess, laboratories, wheelhouse/control room to monitor the various production processes, wardrobe and more, engine room 11 and propulsion system 9. Anchoring system can be spread moored, external or internal turret, buoy or other types of anchoring arrangement known from floating production ships. Loading and unloading systems on board can be cranes, pumps and equipment that a professional known in the art will be familiar with from production ships.

Floating structures such as tankers or bulk carriers are well suited to be modified into production facilities for single-celled microorganisms or communities of microorganisms, multicellular plants and animal and aquatic organisms. By making use of the deck area and cargo volume of such floating devices, a significant number of fermentation, bioreactor and cultivation tanks can be placed very volume and area optimally, in contrast to on land. It may be an important factor in being able to make such production more cost-optimal.

Another alternative embodiment could be to use such facilities for capturing, storing and processing CO₂. Here you can imagine a solution where you get paid for receiving CO₂. Scientists have today succeeded in producing E. coli bacteria in laboratories that consume CO₂. It is therefore likely that within a few years you will be able to have fast-growing bacteria that consume and bind more CO₂ than they release. Here there may be opportunities to combine with photo bioreactors that consume the remaining CO₂ as food for microorganisms such as algae or multicellular plants and animals to produce, for example, Omega 3-rich products, cosmetic products, medicines, soil improvement products or other.

Such a production facility can also be designed to utilize sludge and off-cuts and thereby utilize minerals and elements (phosphorus) that would otherwise be wasted from the farming industry to produce protein, oils and environmentally friendly biohydrogen and biogas, which will help to make the aquaculture industry greener and at the same time create a competitive advantage by reducing production costs related to feed and energy. The gas produced can then be used as a nutrient medium for the cultivation of bacteria.

Such a facility could be anchored in connection with oil installations or near industry associated with significant emissions of CO₂. Here, cement factories, smelters and/or near fish farms can be mentioned. Here, it will be possible to have several synergies based on the proximity of facilities where biological waste substances such as methane, ethane, propane, butane, CO₂ and P are produced and consumed, to name a few.

FIG. 3a, b, c and d show principle sketches of the cargo spaces 7a-c of the embodiment in FIGS. 1 and 2, seen from another side turned 90 degrees from FIGS. 1 and 2. The cargo spaces 7a-c in FIG. 3a, b, c, d shows all the top part 3a of the fermentation unit placed on the deck and with a pipe loop 3b that goes down to the bottom of the storage tank and up again via pipe penetrations in the deck. The pipe loop can go through the bottom 3c and be laid in the double bottom or exposed to the surrounding sea, if it is practical to, for example, free up space inside the tanks. The pipe loop outside the ship will then have to have sufficient strength or be protected so that they are not damaged.

FIG. 3a shows bioreactors 12a, which can be of the tank bioreactor type with stirring or other known types of bioreactors. Tanks if appropriate may be used as tanks for storage of nutrient substrate, sedimentation tanks and/or bulk tanks.

FIG. 3b shows a bioreactor 12b, which can be of the photo bioreactor type. A photo bioreactor shown here uses a light source to grow phototrophic microorganisms/microalgae, which use photosynthesis to generate biomass from light and carbon dioxide.

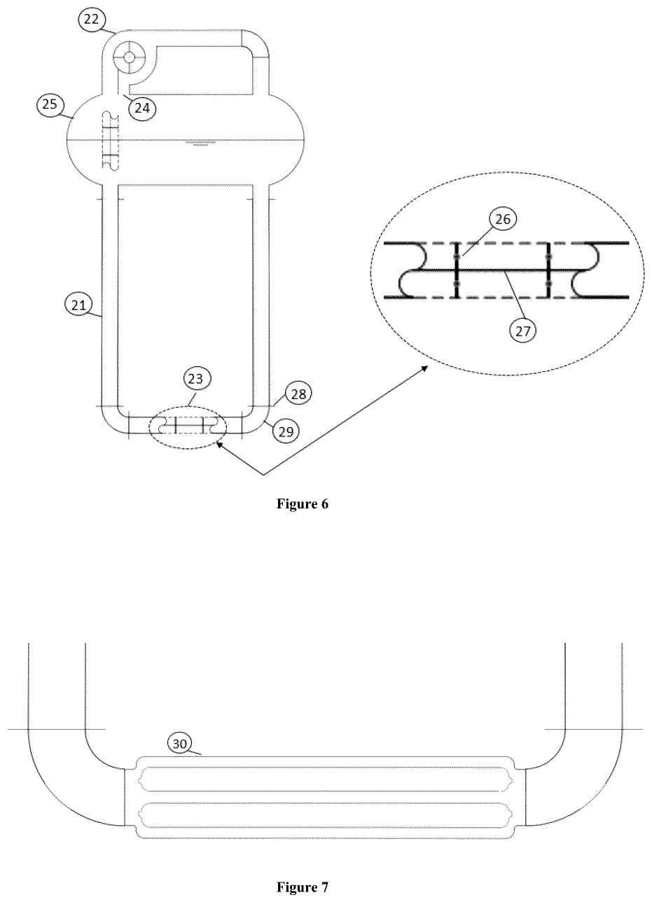

FIG. 3c shows a storage tank filled with liquid 12c. Here one can imagine that these can function as breeding tanks or cultivation tanks for aquatic organisms. These tanks can be open to the surrounding sea or they can be closed systems with water purification and water recycling. Alternatively, they can be a hybrid solution between an open and closed system. Here, the liquid in the cargo tanks may also act as a cooling medium to keep the temperature in the fermentation and bioreactor loops stable. For extra cooling effect, one embodiment can have one or more branch pipes in the lower part of the pipe loop to get a larger heat transfer area as shown in FIG. 7. In order to maintain the correct temperature in fermentation and the bioreactor loop, it may also be possible to utilize the regasification process (the evaporation) of liquefied natural gas for cooling.

Partially or completely utilizing residues from farming as a nutrient source for single-cell protein production provides a significant improvement in aquaculture concepts. This helps to make the production of aquatic organisms more climate-neutral and at the same time contributes to the production of protein that can be turned into fishmeal and omega 3-rich fish feed ingredients.

A solution that FIG. 3c outlines also opens the way to thinking about fish logistics in a new way. Here, it will be possible to transport fish alive over longer distances and thereby contribute to reducing, among other things, air freight and heavy traffic on the road. Until now, such logistics have been difficult to calculate home economically, since there has been no income on the trip back (Back haul). By combining such logistics with protein production, you achieve a continuous income stream that results in lower transport costs. This is a solution that will help to make traditional sea farming even more competitive.

FIG. 3d shows an area that can be located in the hold tank for one or more closed habitats 12d. They will be able to satisfy the strictest requirements for hygiene, temperature, humidity, emissions and so on. Here, it will be possible to cultivate special types of organisms, typically those used for the production of medicines or pharmaceutical products to name a few. One can also imagining placing a combined bioreactor and fuel cell unit for continuous electricity production here.

The solutions described above can of course be combined.

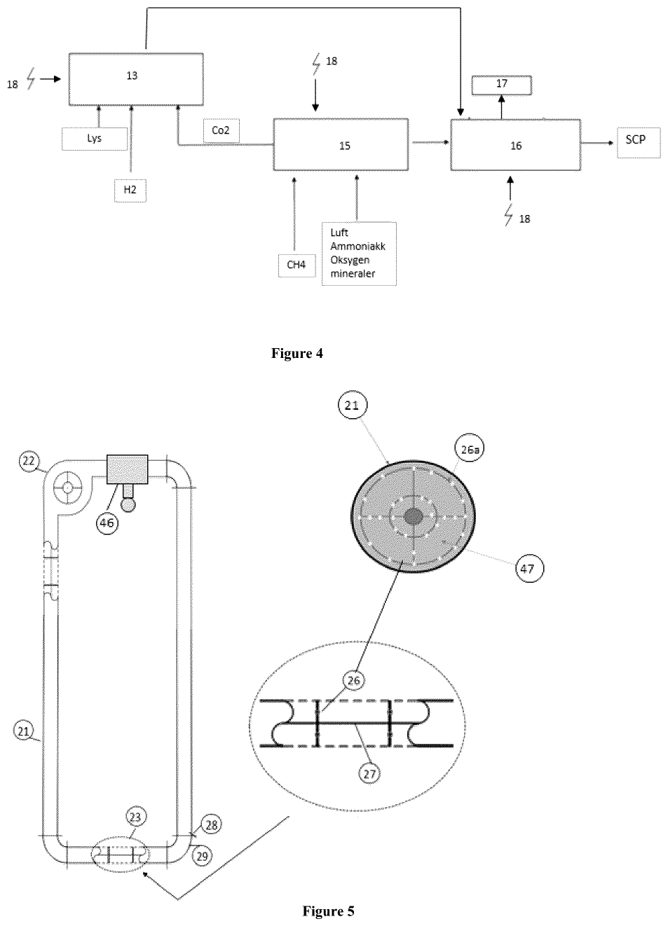

FIG. 4 shows a process flow chart for a climate-neutral way of cultivating and harvest single-celled microorganisms, communities or collections of microorganisms and multicellular plants and animals. Form shows a process consisting of two different biochemical processes. The first process takes place in the fermenter/fermentation unit 15, where methane together with ammonia, minerals and water form a nutrient medium for the cultivation of bacteria. The fermentation process typically produces large amounts of carbon dioxide and in order to make use of this residual product, the intention is to utilize this in another biochemical process, thereby achieving a more climate-neutral production. The off-gases from the fermentation process are stripped and supplied as food into the bioreactor 13, which can be of the photo bioreactor type. This is a type of bioreactor that uses a light source to grow phototrophic microorganisms/microalgae, which use photosynthesis to generate biomass from light and carbon dioxide. Alternatively, bacteria that consume CO₂ Can be cultivated in bioreactors. Supply of energy 18 to operate the processes 13,15,16 can come from renewable energy sources such as offshore wind, hydrogen/fuel cell, sun and or wave power. Alternatively, with fossil combustion with CO₂ capture. The biomass produced 15, 13 can be harvested together with nutrient-rich liquid continuously. In order to obtain protein with the correct solids content, the product is separated and dried in the finishing unit 16. You are then left with a final product that is ready for distribution.

A significant cost associated with the production of single-cell protein and omega 3 products relates to the amount of energy needed to harvest, dry and process the product. It is therefore natural to look to combine with processes that generate waste heat and see if residual heat from these processes can be utilized in the drying process. Such systems can be, for example, a pyrolysis plant. By using renewable energy from, for example, floating wind turbines, the pyrolysis process can be used to split CH₄ into H₂ and black carbon. The hydrogen can be exported, used to generate electricity to operate the vessel and or as a food source for microorganisms. Another residual product is black carbon, which has several areas of application and can be sold. One then achieves a better way to utilize the energy required to dry the protein, etc.

Alternatively, the waste heat can be exported to land or to other offshore installations (FPSOs among others) where the heat energy can be utilized in an optimal way.

FIG. 5 shows a photo bioreactor with an internal tube chain conveyor (23).

It is common today to pump the algae together with growth medium (minerals, phosphorus, nitrate, etc.) around in transparent pipes/tubes designed to provide maximum access to sunlight. In the pipes, carbon dioxide plus minerals are added and oxygen (O₂) is removed. A challenge with these photo reactors is to ensure stable nutrient supply for microalgae growth, optimal light exposure and continuous harvesting. Another challenge is that, over time, a coating will build up on the inside of the pipes, if they not cleaned at regular intervals. Such a coating, if not removed, shields the algae from accessing natural and/or artificial light and by that reduces algae growth. Another challenge with current tube systems is to ensure sufficient agitation so that the algae exposed to as much nutrition and sunlight as possible at all times.

In order to prevent fouling on the inside of the pipes 21, ensure good stirring and provide the best possible nutrient and light exposure, one embodiment thought is to lead algae and nutrient medium inside the pipes 21 by means of a pipe chain conveyor 22,26.27,28. FIG. 5 shows one embodiment of a photo bioreactor with an internal pipe chain conveyor. the chain conveyor consists of a link chain or equivalent 27, with circular but not limited to transport plates/discs 26, which push and or pass the content between the discs inside the pipes 21 in the direction in which it is pulled, alternatively in the opposite direction. These discs 26 may be replaceable. The system is operated and controlled by one or more drive stations 22 and tension station 28, which continuously tension the chain in the conveyor. These can be arranged so that in theory you can have an infinitely long loop with several bends. One can also imagine that the disks could have guide wheels to be guided more easily through the reactor.

In order to achieve optimal light exposure, in one embodiment it is intended to place light sources inside the tubes, for example by equipping the disks with light sources 26a. Here it is also feasible that the disks can be equipped with lights (led but not limited to) with different light spectrums and that they can be turned on and off to achieve optimal growing conditions at all times. It is also possible to have one or more flexible light tubes either continuous or intermittent, in that they are linked/connected to the chain conveyor cable 27 or connected between the disks 26. These light sources which can be led but not limited to can alternatively be powered by small dynamos/electrical generators that generate electricity when the link chain is in motion. Alternatively, the disks can have batteries inside them which are continuously charged by, for example, induction or other types of known energy transfer methods with or without the use of batteries, which are assumed to be known to a person skilled in the art.

By having internal light sources, you can use pipes that are not transparent in whole or in parts. Inside the tubes 21, you can have reflective surfaces which will ensure that the light beams are used optimally. It also makes it possible to use porous cloths inside the pipes to supply CO₂ or other gases. It also makes it possible to place the tubes in places where it is not appropriate or possible to place an external light source. It also makes it possible to insulate the pipes from heat and lack of heat.

An alternative application of the chain conveyor is to pull the discs with the light source against the current. The light source will then be able to expose a larger area, while at the same time stirring the algae medium, for example by ensuring that the disks are not tight and that the nutrient medium is pumped/pushed in the opposite direction. This can also be used in connection with harvesting.

Dissolved iron is known to positively affect the growth of microalgae and the discs can be used to add iron. In one embodiment, one can envisage having disks that contains iron sulphides, and which can emit this inside the proliferation volume. Phosphorus and nitrate are also minerals/nutrients that can be supplied locally in this way to maintain growth conditions.

The use of pipe chain conveyors to transport dry matter is known from the industry, and there are a number of disk solutions that can be adapted to the intended purpose. What is new and innovative, however, is using such pipe chain conveyors to produce microalgae and handle liquid masses. Here are some disk solutions, not exhaustive or limiting.
Disks with sensor technology that measure pH, temperature, algae concentration, linked to a control system that adjusts the speed of the chain conveyor and light spectrum.
Disks with different types of filter for distribution and harvesting of algae.
Discs with light with different light spectrums that can be turned on and off.
Disks with battery/power storage
Discs with segregated volumes, pockets/nets to capture gases, algae
Disks with cooling and or heating properties.
Discs that have built-in propellers that can rotate and thereby create circulation of the medium inside the tube so that you get smaller co2 bubbles that mix more easily in the liquid.

Another application of the chain conveyor is to use it for continuous harvesting of micro algae. One or more of the disks 26 may be equipped with a filter with a specific filter size. A collection unit can be connected to the filter. As the disk lifts out of the nutrient liquid, the excess liquid will decant/run off and we are left with microalgae slush with considerably less liquid content. The harvesting process then takes place by either brushing, blowing and or a flushing system 46 that cleans the filter for micro algae's, and collect and transported the harvested microalgae to a processing station. The harvest process unit ensure that algae film and dirt from the light source being removed. In an alternative embodiment, the disks 26 can be equipped with one or more electrolysis devices where hydrogen and water splits. One can achieve that the hydrogen attracts the algae and carries it up to the surface. Such a system can also be placed in the reactor independently of the disk solution.

The solutions described make it possible to harvest algae continuously and or intermittently during operation, which is important for maintaining production without shutdowns. Harvesting and drying are two significant cost drivers when it comes to harvesting microalgae.

The present invention thus represents a technical solution that solves several challenges associated with today's bioreactors.

It is also possible to use chain conveyors inside the fermentation unit, as shown in FIG. 6. Such system as described here will be particularly suitable for bacteria that consume CO₂, for example we can mention, the Escherichia coli bacteria. Unlike algae, such bacteria grow faster and will be able to consume and tie up larger amounts of CO₂. It is not unimaginable that you can also produce both bacteria and algae in the same reactor. The fact that CO₂ mixes more easily with liquid means that this process will require significantly less energy to dissolve/mix CO₂ in the liquid.

FIG. 6 shows a fermentation reactor with pipe chain conveyor. It is based on the same principles as for the bioreactor described above. Proliferation volume 21 is shown here in an alternative embodiment in combination with a surrounding larger proliferation volume tank (25). a drainage part (24) between (21) to (25) is also shown here. In an alternative embodiment of the fermentation reactor shown in FIG. 6, several drive stations (22) and tensioning devices (28) for the chain conveyor (23) can be included. In this way, several bends (29) can be introduced in the conveyor passage and with this, in theory, an infinitely long pipe loop. It is theoretically possible to fill the entire hold with such a loop.

FIG. 7 shows a fermentation reactor with branch pipe 30. For extra cooling effect, one can have two or more manifolds/branch pipes 30 in the lower part of the pipe loop in one embodiment. One achieve a larger heat transfer area, which again make it possible to maintain steady temperature within the reactor. As a bonus, you may get some temperature transfer to the water inside the tanks, which will be beneficial if you combine it with the breeding of aquatic organisms. Each of the branches may be equipped with suitable equipment such as pumps, valves, nozzles, agitators etc. so that they can work independently and maintenance can be carried out etc.

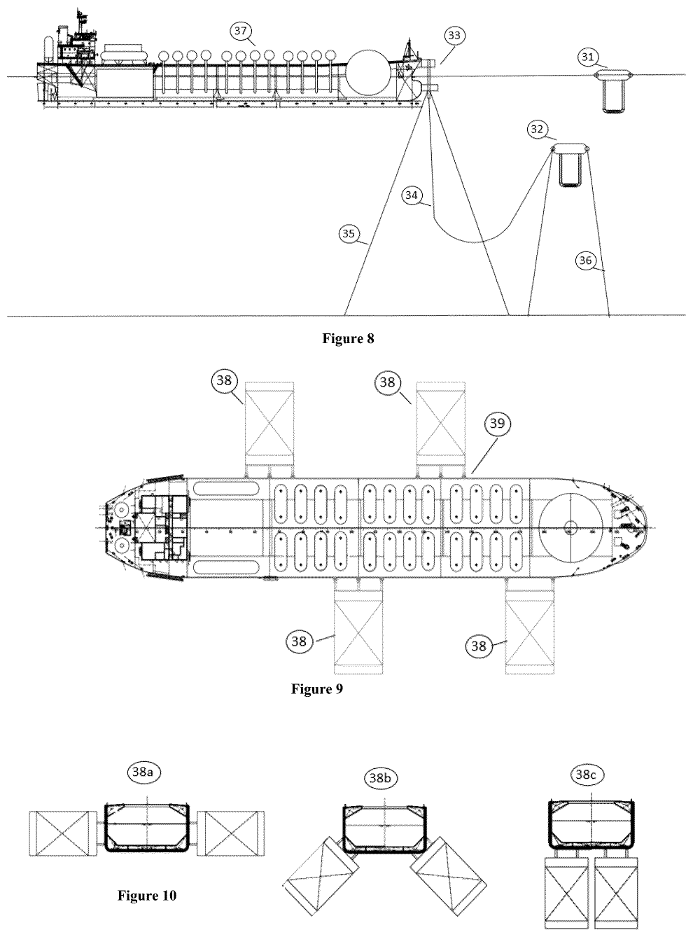

FIG. 8 shows a principle sketch of a self-floating 31 and or a submersible 32 fermentation reactor and or photo bioreactor which is connected to a support vessel 37. The vessel 37 is shown here with an external turret 35 anchoring solution with one or more risers and or umbilical cables 34 that connect the vessel to the fermentation reactors 31,32. The solution is not limited to having a support vessel (ship, barge, farming cage, self-floating structure), as one can just as easily envisage subsea solutions or land plants. The self-floating 31 reactor must be able to be connected by flexible structures with other reactors. You can then place as many units as you need. The self-floating reactor will have one or more buoyancy devices which ensure that the unit is self-floating. Here you can also imagine a solution that makes it possible to tilt the lower loop up to the surface, or raise the entire unit out of the water. Each of these reactors can be equipped with a solar panel. One can also imagine that these can be submerged in the sea or in a cage so that they are less exposed to currents, wind and waves etc . . .

FIG. 9 shows a schematic diagram of a production unit with external rearing cages 38 which are anchored to the hull via specially designed catwalks that fit an H or T-beam, which follow the sides and bottom of the hull and allow the catwalk to move the cage from the starboard side to the port side. In this embodiment, the breeding cage has buoyancy and can have an active and passive ballast system, air pockets when submerged, hoses for underwater feeding and a system to catch dead fish and waste. To dampen the heave movements, the structure that connects the cage to the hull can have shock absorbers that convert movement energy into heat and dampen the relative movement between vessel and breeding cage.

FIG. 10 shows a principle sketch of the production unit shown in FIG. 9 transverse, and shows normal 38a, intermediate 38b and protected condition 38c.

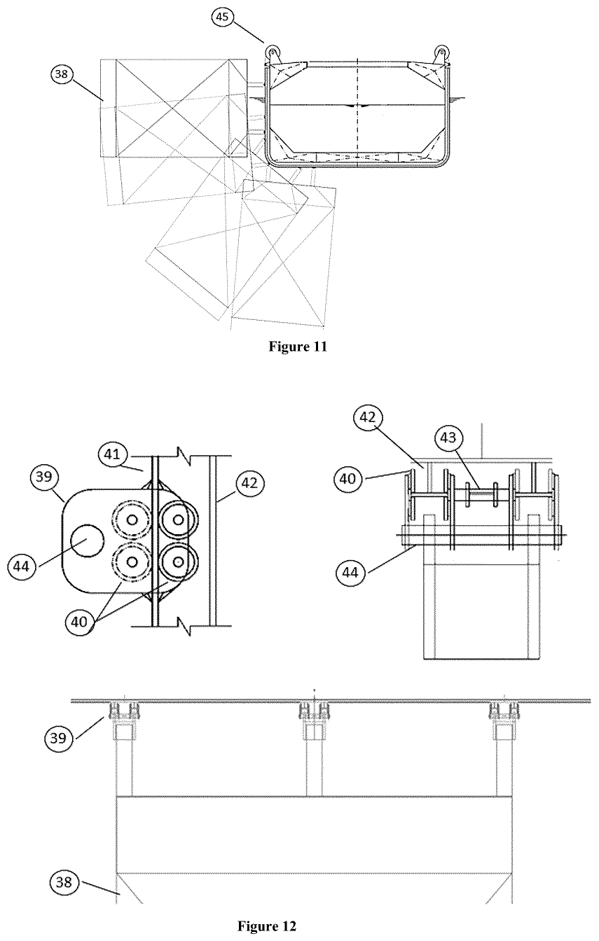

FIG. 11 Shows in principle how the breeding unit can be operated with Winches 45 on deck together with ballasting of the buoyancy units on the breeding pen 38.

To lower the cage 38, the buoyancy tanks are filled with ballast water so that it loses buoyancy and sinks. At the same time as the rearing cage lowers, the running cats along the rails will guide the rearing unit from position 38a to 38b. The winch system can include one or two winches where one releases and one retracts and has the task of unlocking the cage in various positions. In addition, the winches will also assist in pulling the breeding cage around.

When the rearing cage has to come up to the surface again, the process is reversed. Ballast water is displaced or pumped out at the same time as the winch system helps pull the farming unit up to the surface and locks it off. If necessary, you can also have a physical locking pin.

FIG. 12 shows a schematic diagram of the attachment device 39 which connects the rearing unit to the hull of the floating device. The attachment device is a type of trolley with 8 rollers/wheels 40. The wheels have a good rolling function and will be adjustable. It will be able to cover a wide range of beams and profiles 42. To remove any fouling, there are alternatively arranged knives 41 which will scrape away the growth when the running cat moves along the long beam/guide frame 42. The running cat is connected to the rearing unit via a strong bolt 44 which can be removed. You can then easily replace or remove the rearing cage.

1. Production equipment for the production of biological material, comprising: a bioreactor for the cultivation and production of unicellular microorganisms and communities of microorganisms as well as multicellular aquatic plant and animal organisms,
characterized in that the bioreactor has at least one space for the proliferation of unicellular microorganisms, communities of microorganisms, multicellular aquatic plants and animals, which space is in communication with a device for cultivating, transporting and harvesting said microorganisms or aquatic organisms.

2. Production equipment according to claim 1, where the device for cultivating, transporting and harvesting comprises
a chain conveyor (22, 26, 27, 28) inside the proliferation space which can be a pipe loop.

3. Production equipment according to claim 2, where the proliferation space in the form of a tube loop can be open or closed to a larger surrounding volume, e.g. a tank.

4. Production equipment according to claim 1, where the bioreactor comprises branch pipes for moving the contents of the bioreactor to outside the proliferation space, which branch pipes can run inside or outside the bioreactor.

5. Production equipment according to claim 1, where the chain conveyor comprises transport plates in the form of disks .

6. Production equipment according to claim 5, where the disks are equipped with one or more of;
sensor technology that measures pH, temperature and/or algae concentration, linked to a control system that adjusts the speed of the chain conveyor and light spectrum,
different types of filter for distribution and harvesting of algae,
light with different light spectrums and can be turned on and off,
battery and or energy storage,
segregated volumes, pockets/nets to capture gases, algae,
cooling and or heating means,
built-in propellers that can rotate and thereby create circulation of the medium inside the pipe so that you get less CO₂—bubbles that mix more easily in the liquid.

7. Production equipment according to claim 5, where the discs are replaceable, so that you can switch between different functions

8. Production equipment according to claim 6, comprising a unit in which the filters are cleaned for harvesting of algae and a collection unit, and where the algae is led to a processing station.

9. Production equipment according to claim 1, where the bioreactor is equipped with at least one buoyancy body.

10. Production equipment according to claim 1, where the bioreactor is a photo bioreactor.

11. Production equipment according to claim 1, where the bioreactor is a fermenter.

12. Production facility comprising production equipment for the production of biological material according to claim 1, comprehensively at least one fermentation reactor and or at least one bioreactor.

13. Production plant according to claim 12, where the fermentation reactor is supplied with methane, ammonia, minerals and water which form a nutrient medium for the cultivation of bacteria, and where the fermentation process produces carbon dioxide, and the waste gases from the fermentation process are stripped and supplied as food in the bioreactor, which can be of the photo bioreactor type where a light source is used to grow phototrophic microorganisms/microalgae, which use photosynthesis to generate biomass and oxygen from light and carbon dioxide, or alternatively can one cultivate bacteria that consume CO₂ in bioreactors, and where supplied energy, to operate the reactor processes and a drying and separation after-treatment unit, can come from renewable energy sources such as offshore wind, hydrogen/fuel cell, solar and/or wave power.

14. Production plant according to claim 12, where the plant is on land.

15. Production plant according to claim 12, where the plant is located on a floating device such as a vessel, where the fermentation reactor comprises a top part which is placed on the deck of the vessel and has a pipe loop that goes down into the hull of the vessel and up again and where the bioreactor is mainly located down in the hull.

16. Production facility according to claim 15, comprehensive the at least one fermentation reactor, the at least one bioreactor and at least one cultivation tank and/or unit for aquatic organisms, where
cultivation tank is located below the hull of the floating device, where
a cage is placed outside the hull of the floating device and has a guide system along the side of the vessel to lower and raise the cultivation unit.
